(19) United States
(12) Patent Application Publication
(10) Pub. No.: US 2019/0030210 A1
(43) Pub. Date: Jan. 31, 2019

(54) METHODS FOR DETERMINING TISSUE ENGINEERED CONSTRUCT READINESS

(71) Applicant: Cornell University, Ithaca, NY (US)

(72) Inventors: Jill M. Middendorf, Ithaca, NY (US); Lawrence J. Bonassar, Ithaca, NY (US); Lena R. Bartell, Ithaca, NY (US); Itai Cohen, Ithaca, NY (US)

(21) Appl. No.: 16/025,709

(22) Filed: Jul. 2, 2018

Related U.S. Application Data
(60) Provisional application No. 62/527,260, filed on Jun. 30, 2017.

(51) Int. Cl. A61L 27/36; C12N 5/00

(52) U.S. Cl. CPC A61L 27/3691; A61L 27/3695; C12N 5/0062

(57) ABSTRACT

The present invention relates to methods of determining if a tissue engineered construct is ready for implantation. In one aspect the method involves providing a tissue engineered construct comprising a scaffold having pores, analyzing the tissue engineered construct for buckling of pores, and determining whether the tissue engineered construct is ready for implantation based on the analysis. In another aspect, the invention relates to a method that involves non-destructive methods for determining whether a tissue engineered construct is ready for implantation.

This application claims the benefit of a U.S. Provisional Patent Application Ser. No. 62/527,260, filed on Jun. 30, 2017, which is hereby incorporated by reference in its entirety.

This invention was made with government support under grant number AR069977 awarded by the National Institutes of Health and grant numbers 1536463 and 1650441 awarded by the National Science Foundation. The government has certain rights in the invention.

### FIELD OF THE INVENTION

The present invention is directed to methods for determining tissue engineered construct readiness for implantation.

### BACKGROUND OF THE INVENTION

The poor intrinsic repair capabilities of certain tissues, for example native articular cartilage, has driven interest in tissue engineered constructs. Several tissue engineered cartilage techniques embed cells onto a 3D porous scaffold that allows for tissue growth (Brittberg, “Cell Carriers As The Next Generation Of Cell Therapy For Cartilage Repair: A Review Of The Matrix-Induced Autologous Chondrocyte Implantation Procedure,” Am. J. Sports Med. 38:1259-71 (2010) and Hunziker et al., “An Educational Review Of Cartilage Repair: Precepts & Practice—Myths & Misconceptions—Progress & Prospects,” Osteoarthr. Cartil. 23:334-50 (2015)). Such techniques have been successful clinically (Brittberg, “Cell Carriers As The Next Generation Of Cell Therapy For Cartilage Repair: A Review Of The Matrix-Induced Autologous Chondrocyte Implantation Procedure,” Am. J. Sports Med. 38:1259-71 (2010) and Kon et al., “Matrix Assisted Autologous Chondrocyte Transplantation For Cartilage Treatment: A Systematic Review,” Bone Joint Res. 2:18-25 (2013)), filling the defect, maintaining cell viability, and inducing new matrix growth (Willers et al., “Autologous Chondrocyte Implantation with Collagen Bioscaffold for the Treatment of Osteochondral Defects in Rabbits,” Tissue Eng. 11:1065-76 (2005)). However, biological markers alone are not enough to understand the success of such implants. Notably, FDA guidance advises measuring the mechanical properties of these implants to better understand their function (U.S. Food and Drug Administration, “Guidance for Industry: Preparation of IDEs and INDs for Products Intented to Repair or Replace Knee Cartilage,” www.FDA.gov (2011)).

The global compressive properties of tissue engineered cartilage implants using porous cell seeded 3D scaffolds have been well documented. A variety of scaffold materials, scaffold shapes, cell types, and growth conditions affect the compressive properties of the constructs (Temenoff et al., “Injectable Biodegradable Materials for Orthopedic Tissue Engineering,” Biomaterials 21:2405-12 (2000)). The compressive modulus vary widely from about 2% to nearly 90% of the native values (Cigan et al., “High Seeding Density of Human Chondrocytes in Agarose Produces Tissue-Engineered Cartilage Approaching Native Mechanical and Biochemical Properties” J. Biomech. 49:1909-17 (2016); Paschos et al., “Functional Properties of Native And Tissue-Engineered Cartilage Toward Understanding the Pathogenesis of Chondral Lesions at the Knee: A Bovine Cadaveric Study,” J. Orthop. Res 35(11):2452-64 (2017); Peng et al., “Surface Zone Articular Chondrocytes Modulate the Bulk and Surface Mechanical Properties of the Tissue Engineered Cartilage,” Tissue Eng. Part A 20(23-24):3332-41 (2014); Rosenzweig et al., “Cartilaginous Constructs Using Primary Chondrocytes from Continuous Expansion Culture Seeded in Dense Collagen Gels,” Acta Biomater. 9:9360-69 (2013); and Tang et al., “Feasibility of Autologous Bone Marrow Mesenchymal Stem Cell-Derived Extracellular Matrix Scaffold for Cartilage Tissue Engineering,” Artif. Organs 37:E179-90 (2013)). In many studies, an increase in proteoglycans (GAG) content was correlated with an improved compressive moduli (Griffin et al., “Mechanical Properties and Structure-Function Relationships in Articular Cartilage Repaired Using Igf-I Gene-Enhanced Chondrocytes,” J. Orthop. Res. 34:149-53 (2016); Klein et al., Depth-Dependent Biomechanical and Biochemical Properties of Fetal, Newborn, and Tissue-Engineered Articular Cartilage,” J. Biomech. 40:182-90 (2007); Mauck et al., “Influence Of Seeding Density and Dynamic Deformational Loading on the Developing Structure/Function Relationships of Chondrocyte-Seeded Agarose Hydrogels,” Ann. Biomed. Eng. 30:1046-56 (2002); and Middendorf et al., “Mechanical Properties and Structure-Function Relationships of Human Chondrocyte-Seeded Cartilage Constructs After In Vitro Culture,” J. Orthop. Res. 1-9 (2017)). However, the mechanical interactions between newly deposited GAG and the porous scaffold are not well understood.

A number of studies have linked the compressive behavior of porous scaffold materials to their microscale structure. Under small compressive strains, porous materials such as honeycomb scaffolds, exhibit linear stress-strain behavior associated with a slight bending of the pore walls (Gibson et al., Cellular Solids: Structure and Properties, 2nd ed. Cambridge University Press, Cambridge, UK (1997)). At higher strains, the stress-strain curve enters a plateau region, where the walls become unstable and buckle. The onset of buckling in these porous structures can be changed by increasing wall thickness or depositing material within the pores (Gibson et al., Cellular Solids: Structure and Properties, 2nd ed. Cambridge University Press, Cambridge, UK (1997) and Slivka et al., “Porous, Resorbable, Fiber-Reinforced Scaffolds Tailored for Articular Cartilage Repair” Tissue Eng. 7:767-80 (2001)). Both of these phenomena strengthen the porous structure and may thus explain changes in the compressive mechanics of tissue engineered cartilage due to matrix deposition into pores or on scaffold surfaces. This matrix synthesis is expected to locally reinforce the scaffold and thus increase the strain needed to induce buckling. However, the amount of matrix synthesis required to reinforce the scaffold to delay buckling is unknown.

In addition to the total amount of matrix deposition, the location of matrix in the scaffold may also influence the local buckling. Matrix deposition on porous scaffolds is highly heterogeneous (Klein et al., “Depth-Dependent Biomechanical and Biochemical Properties Of Fetal, Newborn, and Tissue-Engineered Articular Cartilage,” J. Biomech. 40:182-90 (2007); Krase et al., “BMP Activation and Wnt-Signalling Affect Biochemistry and Functional Biomechanical Properties of Cartilage Tissue Engineering Constructs,” Osteoarthr. Cartil. 22:284-92 (2014); and Middendorf et al., “Mechanical Properties and Structure-Function Relationships of Human Chondrocyte-Seeded Cartilage Constructs after In Vitro Culture,” J. Orthop. Res. 1-9 (2017)). Previously, extensive matrix deposition on the outside edges of the scaffold with less deposition on the inside pore surfaces has been shown. Scaffold pores with more matrix deposition reinforce the scaffold, while scaffold pores with little or no matrix deposition may create local weaknesses. Local structural variations caused by matrix deposition will affect the global compressive properties. However, the relationship between microscale compressive mechanics, the GAG content, and the onset of buckling in tissue engineered constructs has not been studied. Techniques to measure the local strain in native and tissue engineered cartilage have been recently developed (Buckley et al., “High-Resolution Spatial Mapping of Shear Properties in Cartilage,” J. Biomech. 43:796-800 (2010) and Buckley et al., “Mapping the Depth Dependence of Shear Properties in Articular Cartilage,” J. Biomech. 41:2430-37 (2008)), but these techniques have not been applied to identifying local compressive mechanics and buckling.

In previous studies, the global compressive modulus of cultured implants improved with increased culture and improved with increased GAG content (Middendorf et al., “Mechanical Properties and Structure-Function Relationships of Human Chondrocyte-Seeded Cartilage Constructs after In Vitro Culture,” J. Orthop. Res. 1-9 (2017)). However, the microscale mechanism behind this improvement is unknown.

Many studies have measured the global compressive properties of tissue engineered (TE) cartilage grown on porous scaffolds. Such scaffolds are known to exhibit strain softening due to local buckling under loading. As matrix is deposited onto these scaffolds, the global compressive properties increase. However, the relationship between the amount and distribution of matrix in the scaffold and local buckling is unknown.

The present invention is directed to overcoming these and other deficiencies in the art.

### SUMMARY OF THE INVENTION

A first aspect of the present invention is directed to a method of determining if a tissue engineered construct is ready for implantation. This method involves providing a tissue engineered construct comprising a scaffold having pores, analyzing the tissue engineered construct for buckling of pores, and determining whether the tissue engineered construct is ready for implantation based on said analyzing.

A second aspect of the present invention is directed to a method of determining if a tissue engineered construct is ready for implantation. This method involves providing a tissue engineered construct comprising a scaffold having pores; analyzing the tissue engineered construct in a non-destructive manner; and determining whether the tissue engineered construct is ready for implantation.

Engineered tissue constructs are used as a solution to tissue damage and tissue loss. Tissue engineering typically involves the use of cells, scaffolds, and stimulating factors, either alone or in various combinations. To be ready for implantation, tissue engineered constructs must have mechanical properties that mimic the tissue which they are intended to replace. See, e.g., Little et al., “Mechanical Properties of Natural Cartilage and Tissue-Engineered Constructs,” Tissue Engineering Part B: Reviews 17(4):213-27 (2011), which is hereby incorporated by reference in its entirety. For example, tissue engineered cartilage constructs preferably mimic the properties and structure of native cartilage. Thus, upon implantation, the construct must be able to maintain its structural integrity under compressive forces.

Production of tissue engineered constructs requires quality control measures to ensure that constructs are ready for implantation. Traditional quality control procedures involve costly destructive sampling and/or suffer from lack of precision. FDA guidance suggests what to measure, but gives no target values. As such, what anyone might pick as their own threshold for quality control of compressive modulus (for example) is arbitrary. In general, the field has viewed the progression of these constructs as fairly linear (e.g. 20% more GAG should mean 20% higher modulus). However, it has now been discovered that this is not true. There are discreet/quantum changes in construct performance based on amount of matrix filling the pores. At some point, fill is sufficient to prevent a pore from buckling and the performance of the construct would be expected to be much better. Recognizing that such transitions exist is novel, important, non-obvious, and useful. The present invention provides ways to measure this through mechanical testing and vibrational spectroscopy, and track this transition. The present invention is directed to overcoming existing difficulties and provides novel procedures for determining whether tissue engineered constructs are ready for implantation.

For example, the present invention pertains to how GAG deposition influences the microscale compressive properties of tissue engineered cartilage with respect to scaffold buckling. In one example of one embodiment of the present invention, an implant similar to NeoCart®, made of a 3D collagen type I honeycomb scaffold seeded with human chondrocytes, was examined. NeoCart® is currently in advanced human clinical trials and has shown good integration and defect filling (Crawford et al., “Neocart, an Autologous Cartilage Tissue Implant, Compared with Microfracture for Treatment of Distal Femoral Cartilage Lesions: An FDA Phase-II Prospective, Randomized Clinical Trial after Two Years,” J. Bone Joint Surg. Am. 94:979-89 (2012) and Crawford et al, “An Autologous Cartilage Tissue Implant Neocart for Treatment of Grade III Chondral Injury to the Distal Femur: Prospective Clinical Safety Trial at 2 Years,” Am. J. Sports Med. 37:1334-43 (2009), which are hereby incorporated by reference in their entirety).

The present invention identifies the local strain and local scaffold buckling in tissue engineered constructs as a function of culture times and identifies the relationship between local buckling and GAG content. To further understand human tissue engineered construct maturity, the biochemical composition is related to construct buckling and the local mechanical properties.

The present invention addresses how local strain and construct buckling in human TE constructs changes over culture times and GAG content. Confocal elastography techniques and digital image correlation (DIC) were used to measure and record buckling modes and local strains. Receiver operating characteristic (ROC) curves were used to quantify construct buckling. Results from ROC analysis are placed into Kaplan-Meier survival function curves to establish the probability that any point in a construct buckles. These analysis techniques reveal the presence of buckling at early time points, but bending at later time points. An inverse correlation is observed between the probability of buckling and the total GAG content of each construct. This data suggests that increased GAG content prevents the onset of construct buckling and improves the microscale compressive tissue properties. This increase in GAG deposition leads to enhanced global compressive properties by prevention of microscale buckling.

### BRIEF DESCRIPTION OF THE DRAWINGS

FIGS. 1A-1C provide an overview of sample preparation and buckling analysis according to one embodiment of the present invention. FIG. 1A depicts: (Box A1) constructs were cut in half then stained with dichlorotriazinylamino fluorescein (DTAF), (Box A2) videos were recorded of construct compression to 10% axial strain, (Box A3) videos were analyzed using digital image correction (DIC) software, (Box A4) buckling modes were visually identified and labeled both globally and locally. FIG. 1B depicts: (Box B1) the global buckling analysis identified the 50^{th}, 75^{th}, and 85^{th} percentile of strain/strain rate histograms (one strain/strain rate histogram per construct), (Box B2) recorded values and visual buckling information were placed into receiver operating characteristic (ROC) curves to identify the best threshold, (Box B3) the global buckling threshold was verified with the remaining 17 constructs. FIG. 1C depicts: (Box C1) the local buckling analysis was completed on 6 constructs by examining the buckling mode and strain/strain rate at each location in the grid, (Box C2) data was then placed into an ROC curve to determine the best threshold, (Box C3) the threshold was verified by examining 2 additional constructs.

FIGS. 2A-2D show the axial strain of individual constructs at multiple time points while under axial compression. FIG. 2A is a series of images showing that at 1 week in culture axial compression videos show areas of high axial strain and compressive failure (inserts indicate areas of buckling, white arrows indicate fibers that were originally straight, but are now kinked or buckled). FIG. 2B is a series of images showing that at 3 weeks in culture construct buckling is less pronounced than in the 1 week construct and that buckling began at higher axial strains than 1 week constructs. FIG. 2C is a series of images showing that after 5 weeks constructs begin to form a thin surface layer of proteoglycans with no construct buckling. FIG. 2D is a series of images showing that at seven weeks constructs show resistance to compressive failure and a compliant surface associated with a thicker layer of proteoglycans.

FIGS. 3A-3C provide an overview of one embodiment of ROC analysis. FIG. 3A is a graph showing a perfect ROC and a ROC with random data. The best threshold value can be found using the “closest to the top left” method, which finds the value that minimizes the distance to the top left using least squares analysis. FIG. 3B is a graph showing that after plotting the ROC curves the area under the curve (AUC) can be calculated and compared for each strain and strain rate examined at each percentile. The highest AUC identifies the parameter that fits the ROC the best. In this study the transverse strain (E_{yy}) at the 75^{th} percentile provides the best fit to the ROC curve. FIG. 3C is a graph showing that when the distance to the top left of the ROC curve is plotted versus all possible threshold values, the best threshold value of E_{yy}=3.2% can be easily identified. Collectively, the data depicted in FIGS. 3A-3C indicate that when the top 25% of transverse strains exceed 3.2%, the construct will experience local buckling.

FIG. 4 is a series of graphs showing axial strain versus depth of individual constructs at multiple time points while under axial compression. After 1 week in culture, axial compression videos show random areas of high axial strain that correspond to the areas of buckling. All constructs exhibited buckling after 1 week in culture. After 7 weeks, constructs show resistance to buckling. A compliant surface zone associated with a thicker layer of proteoglycans was revealed. All constructs exhibited bending. At 3 and 5 weeks in culture, constructs exhibited characteristics of both 1 and 7 week constructs.

FIGS. 5A-5C depict results of one embodiment of ROC analysis pertaining to an embodiment of the methods of the present invention. FIG. 5A is an image showing a portion of the displacement grid on a buckled construct. Each grid point was visually inspected and labeled Y (yes buckling) or N (no bending). FIG. 5B shows that after creating ROC curves for each variable tested the AUC can be compared. Both the axial strain (Eₓₓ) and transverse strain (E_{yy}) fit the ROC curve well. FIG. 5C shows that analysis of the two data sets that fit the ROC the best are plotted using the “least squares method.” This method found that the transverse strain provided a better threshold value (E_{yy}) than the axial strain (Eₓₓ).

FIGS. 6A-6B depict Kaplan Meier survival function analysis. FIG. 6A shows that using a Kaplan Meier survival function the probability that any grid point on a construct at a specific time point can be calculated and plotted versus the bulk axial strain. The curves for each time point are statistically different from each other with 1 week constructs being the most likely to undergo buckling and 7 week constructs being the least likely to undergo buckling. FIG. 6B shows that when the probability of buckling on a given construct is plotted vs the bulk proteoglycan (GAG) content of that construct, linear correlations are observed. The strongest linear correlation occurred at 10% bulk strain (p=0.008) and the weakest correlation occurred at 2.5% bulk axial strain (p=0.06).

FIGS. 7A-7F depict buckling analysis. FIG. 7A is a representative image showing buckled and non-buckled constructs with corresponding Safranin-O staining and a DIC strain map. FIG. 7B is a representative Safranin-O stained image showing the 2 tissue types (TisType) contained on a construct. FIG. 7C is a plot depicting the probability of any point in a construct buckling based on applied strain and total GAG content. FIG. 7D is a graph showing the relative depth dependent concentration of collagen (Col) and aggrecan (Ag) (zero represents interface between tissue types. FIG. 7E shows the correlation of the depth dependent collagen concentration vs shear modulus. FIG. 7F shows aggrecan concentration vs. shear modulus.

FIGS. 8A-8D depict representative images of safranin-O staining of the cross section of tissue engineered constructs. FIG. 8A shows that at early time points, constructs are collagen honeycomb structures with little GAG. FIG. 8B shows that as constructs mature, GAG content is highly concentrated on the outside edges of the scaffold. FIG. 8C shows that GAG content may line the inner pores of the scaffold. FIG. 8D shows that GAG content may fill the inner pores of the scaffold.

### DETAILED DESCRIPTION OF THE INVENTION

One aspect of the present invention relates to a method of determining if a tissue engineered construct is ready for implantation. This method involved providing a tissue engineered construct comprising a scaffold having pores; analyzing the tissue engineered the construct for buckling of pores; and determining whether the tissue engineered construct is ready for implantation based on said analyzing.

As used herein, a “tissue engineered construct” (TEC) refers to a construct that is produced by seeding living cells on a natural or synthetic extracellular substrate and culturing them to create an implantable tissue. The extracellular substrate onto which the living cells are seeded is referred to as a “scaffold.” As used herein, culturing cells refers multiplying the cells and/or inducing them to produce extracellular matrix (“ECM”). In one embodiment, TECs analyzed according to methods of the present invention may comprise three-dimensional scaffolds which comprise an interconnected network of “pores.” As used herein, “buckling” of pores refers to the physical phenomenon where the walls of the scaffold's pores become unstable. Buckling is distinct from bending, in which the pore walls deform but do not become unstable. Buckling of pores is also referred to as “local buckling.” TECs can also be described as buckling on a global level (i.e., the construct can be described as buckled if it exhibits local buckling).

In one embodiment of the present invention, providing a TEC comprises seeding cells onto a scaffold and culturing the cells to form a tissue engineered construct.

Tissue engineered constructs of this and all other aspects of the present invention include, but are not limited to, tissue engineered cartilage constructs, tissue engineered bone constructs, tissue engineered tendon constructs, tissue engineered ligament constructs, and tissue engineered heart valve constructs.

The tissue engineered constructs of this and all other aspects of the present invention can be engineered for implantation, e.g., into an animal. In one embodiment, the tissue engineered construct is a human tissue engineered construct, meaning the TEC is intended for implantation into a human. In another embodiment, the tissue engineered construct is intended for implantation into a non-human animal, for example a mammal, such as a pig, horse, cow, cat, or dog.

The scaffolds of the tissue engineered constructs of this and all other aspects of the present invention include any scaffold now known or later developed. In one embodiment the scaffold is made of a material that is biodegradable and/or biocompatible.

In certain embodiments of this and all other aspects of the present invention, the scaffold is produced via gas foaming/particulate leaching, thermally induced phase separation, electrospinning, 3D printing, selective laser sintering, stereolithography, or fused deposition modeling.

In certain embodiments of this and all other aspects of the present invention, the scaffold material comprises a synthetic material. Exemplary synthetic materials include, but are not limited to, ceramics such as hydroxyapatite and tri-calcium phosphate, synthetic polymers such as polystyrene, poly-l-lactic acid (PLLA), polyglycolic acid (PGA), poly-dl-lactic-co-glycolic acid (PLGA), polyurethanes, polyphosphazenes, polyanhydrides, polyorthoesters, polyethylene glycol (PEG), hydroxyapatite (HAP), and combinations thereof.

In one embodiment, the scaffold material comprises a natural polymer. Exemplary natural polymers include, but are not limited to, collagen, proteoglycans, alginate-based substrates, chitosan, gelatin, and combinations thereof.

In one embodiment, the scaffold material is a honeycomb scaffold. In one embodiment, the scaffold material is a collagen honeycomb scaffold.

In one embodiment, the pores of the scaffold are about 10 μm to about 500 μm in diameter, or about 200 μm to 400 μm, or about 250 μm to about 350 μm, or about 300 μm.

Suitable cell sources for tissue engineered constructs include mammalian cells, e.g., human cells, equine cells, porcine cells, feline cells, and/or canine cells. In one embodiment, the cells are human cells. The cells may be autologous, allogenic, zenogenic, or syngenic. Cells may include both primary and secondary cells, and both stem and differentiated cells. Stem cells include, for example, embryonic, mesenchymal, embryonic-derived mesenchymal, amniotic-fluid derived, and dental pulp stem cells. Differentiated cells include, for example, chondrocytes, bone marrow cells, tenocytes, fibrocytes, valvular endothelial cells, and valvular interstitial cells. Chondrocytes include, without limitation, articular chondrocytes, nasal chondrocytes, tracheal chondrocytes, meniscal chondrocytes, and aural chondrocytes. These also include, for example, mammalian chondrocytes, e.g., human chondrocytes, equine chondrocytes, porcine chondrocytes, feline chondrocytes, and canine chondrocytes. In one embodiment, the tissue engineered construct comprises primary chondrocytes. Bone marrow cells include, for example, mammalian bone marrow cells, e.g., human bone marrow cells, equine bone marrow cells, porcine bone marrow cells, feline bone marrow cells, and canine bone marrow cells. Tenocytes include, for example, mammalian tenocytes, e.g., human tenocytes, equine tenocytes, porcine tenocytes, feline tenocytes, and canine tenocytes. Fibrocytes include, for example, mammalian fibrocytes, e.g., human fibrocytes, equine fibrocytes, porcine fibrocytes, feline fibrocytes, and canine fibrocytes. Valvular endothelial cells include, for example, mammalian valvular endothelial cells, e.g., human valvular endothelial cells, equine valvular endothelial cells, porcine valvular endothelial cells, feline valvular endothelial cells, and canine valvular endothelial cells. Valvular interstitial cells include, for example, mammalian valvular interstitial cells, e.g., human valvular interstitial cells, equine valvular interstitial cells, porcine valvular interstitial cells, feline valvular interstitial cells, and canine valvular interstitial cells.

Tissue engineered constructs may comprise a single cell type or more than one cell type.

In one embodiment, the TEC is a human tissue engineered cartilage construct seeded with human chondrocytes.

In one embodiment, analyzing the TEC for buckling of pores involves compressing the TEC and detecting whether pores of the scaffold of the TEC buckled.

Compressing the TEC may involve, according to one embodiment, placing the TEC under axial compression. The axial compression can be at a level of at least 1%, 2%, 3%, 4%, 5%, 6%, 7%, 8%, 9%, 10%, 11%, 12%, 13%, 14%, 15%, 16%, 17%, 18%, 19%, or 20%.

Compressing the TEC may involve, according to another embodiment, controlling the load and/or stress associated with compressing the construct. In one embodiment, axial stress can reach values of approximately 10-1000 kPa, which correlates to a load of approximately 0.0283 N to 28.3 N.

In one embodiment, buckling and bending of pores can be detected and/or distinguished visually. In another embodiment, buckling and bending can be detected and/or distinguished based on statistical analyses derived from visual observations.

In one embodiment, detecting whether pores of the scaffold of the TEC buckled is involves measuring the microscale axial, transverse, and shear strains and axial, transverse, and shear strain rates on a grid using digital imaging correction to identify a buckling threshold. In one embodiment, the grid size is between 10 μm and 500 μm, or 50 μm and 450 μm, or 100 μm and 400 μm, or 150 μm and 350 μm, or 200 μm and 300 μm, or about 350 μm. In another embodiment, the grid is a 78.7 μm grid, or the grid size ranges from 10 μm to 250 μm in each direction (or, e.g., 50 μm to 200 μm, 100 μm to 150 μm, or about 125 μm).

In one embodiment, the microscale axial, transverse, and shear strains and axial, transverse, and shear strain rates are determined statistically. For example, the microscale axial, transverse, and shear strains and axial, transverse, and shear strain rates are determined by interpolating the microscale displacements using a finite element shape function.

In one embodiment, buckling and bending of pores is detected using an imaging technique. Suitable imaging techniques include, without limitation, magnetic resonance imaging (MRI), computed tomography (CT), ultrasound, optical coherence tomography, and combinations thereof.

In one embodiment, a buckling threshold is identified and applied to classify the TEC as buckled or not buckled. A buckling threshold may be identified statistically by analyzing a group of TECs. The threshold can be identified by applying the “closest to the top left’ method to a receiver operating curve (ROC) analysis to the group of TECs. The ROC curve can be constructed using histogram strain and strain rate percentiles. Analyzing the TEC for buckling of pores may also involve comprises applying a previously identified threshold to newly provided TECs. In one embodiment, buckling is identified by analysis of a stress strain curve and analysis of a force displacement curve.

In one embodiment of the present invention, it is determined that a TEC is ready for implantation if no buckling of the pores of the scaffold of the TEC was detected. In another embodiment, a TEC is ready for implantation if the probability of buckling is less than 25%. For example, a TEC is ready for implantation if the probability of buckling is equal to or less than 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%.

In another embodiment, analyzing the TEC for buckling of pores comprises measuring bulk proteoglycan content. In this embodiment, bulk proteoglycan content is used to determine the probability that the construct will buckle when compressed. For example, the probability of buckling is 20% or less if the proteoglycan concentration is 75 μg/mg (dry weight) under 10% axial strain.

In one embodiment, the method of determining if a tissue engineered construct is ready for implantation involves providing a plurality of TEC and analyzing one or more of these TECs, but not all of them, so as to preserve one or more TECs for implantation.

According to this embodiment, the plurality of tissue engineered constructs may have been created under the same conditions, (e.g., are of the same “lot”) and thus, represent biological replicates presumed to share mechanical characteristics. As such, the implant readiness of the plurality can be inferred by determining whether one or more of the replicates is ready for implantation. Thus, it will be understood that the methods for determining the implant readiness of a tissue engineered construct as described for the first aspect of this invention can be used to determine whether the plurality of tissue engineered constructs of this aspect of the present invention is ready for implantation.

Another aspect of the present invention is directed to a method of determining if a tissue engineered construct is ready for implantation. This method involves providing a tissue engineered construct comprising a scaffold having pores; analyzing the tissue engineered construct in a non-destructive manner; and determining whether the tissue engineered construct is ready for implantation based on said analyzing.

According to this aspect of the present invention, the TEC is analyzed in a non-distractive manner. In one embodiment, this involves detecting biochemical compositional data and determining the probability of whether pores of the scaffold of the tissue engineered construct will buckle when compressed. The biochemical compositional data may be selected from any one or more of total proteoglycan content, total collagen content, total aggrecan content, microscale proteoglycan content, microscale collagen content, and microscale aggrecan content.

Any suitable method for measuring the biochemical compositional data can be used. Suitable methods for measuring microscale biochemical compositional data includes, for example, vibrational spectroscopy including, but not limited to, Fourier Transform infrared spectroscopy (FTIR), Raman, and near infrared spectroscopy (NIR). Microscale biochemical compositional data in the range of about 50 μg/mg to 150 μg/mg aggrecan (dry weight) located in scaffold pores of approximately 200-400 μm give an indication that buckling is prevented at global strains up to 10%.

In one embodiment, determining the probability of whether pores of the scaffold of the tissue engineered construct will buckle when compressed comprises characterizing the tissue engineered construct as having a probability of buckling of 25% or less determining the probability of whether pores of the scaffold of the tissue engineered construct will buckle when compressed comprises characterizing the tissue engineered construct as having a probability of buckling of equal to or less than 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%.

In one embodiment, tissue engineered constructs are characterized as ready for implantation if the probability that the pores of the scaffold of the tissue engineered construct will buckle when compressed is less than 25%. In one embodiment, tissue engineered constructs are characterized as ready for implantation if the probability that the pores of the scaffold of the tissue engineered construct will buckle when compressed equal to or less than 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1%.

### EXAMPLES

### Example 1

### Tissue Construct Preparation

Human tissue engineered cartilage constructs were prepared as described previously (Crawford et al., “Neocart, An Autologous Cartilage Tissue Implant, Compared With Microfracture For Treatment Of Distal Femoral Cartilage Lesions: An FDA Phase-II Prospective, Randomized Clinical Trial After Two Years,” J. Bone Joint Surg. Am. 94:979-89 (2012) and Crawford et al, “An Autologous Cartilage Tissue Implant Neocart for Treatment of Grade III Chondral Injury to the Distal Femur: Prospective Clinical Safety Trial at 2 Years,” Am. J. Sports Med. 37:1334-43 (2009), which are hereby incorporated by reference in their entirety). Briefly, cadaveric normal human cartilage tissue from the femoral condyles of a 28 year old male was obtained under protocol from National Disease Research Interchange (NDRI, Philadelphia, Pa.), then processed by enzymatic digestion with collagenase (Worthington Biochemical, Lakewood, N.J.) to yield chondrocytes. Chondrocytes were isolated, expanded in DMEM/F12 culture medium containing 10% fetal bovine serum (Gibco, Thermo Fisher Scientific, Waltham, Mass.) through passage 1 at 37° C. under 5% CO₂, suspended in a 3 mg/mL type I collagen solution (PureCol, Advanced Biomatrix, San Diego, Calif.) at a concentration of 5×10⁶ cells/mL, and seeded into approximately 6 mm diameter by 1.5 mm thick type 1 collagen honeycomb scaffolds (Itoh et al., “A Honeycomb Collagen Carrier for Cell Culture as a Tissue Engineering Scaffold,” Artific. Organs 25(3):213-217 (2001) (Koken, Tokyo, Japan). Both the scaffold and the collagen solution were produced from bovine. Constructs were incubated at 37° C., 5% CO₂, and 2% O₂ in static culture with media changes at regular intervals. Constructs were removed from culture at multiple stages of development (1, 3, 5, and 7 weeks post seeding) and stored at −20° C. A total of 22 constructs were produced by this process and allocated for compression testing described below, with 4-7 constructs tested at each time point (1, 3, 5, and 7 weeks).

### Example 2

### Compressive Strain Mapping

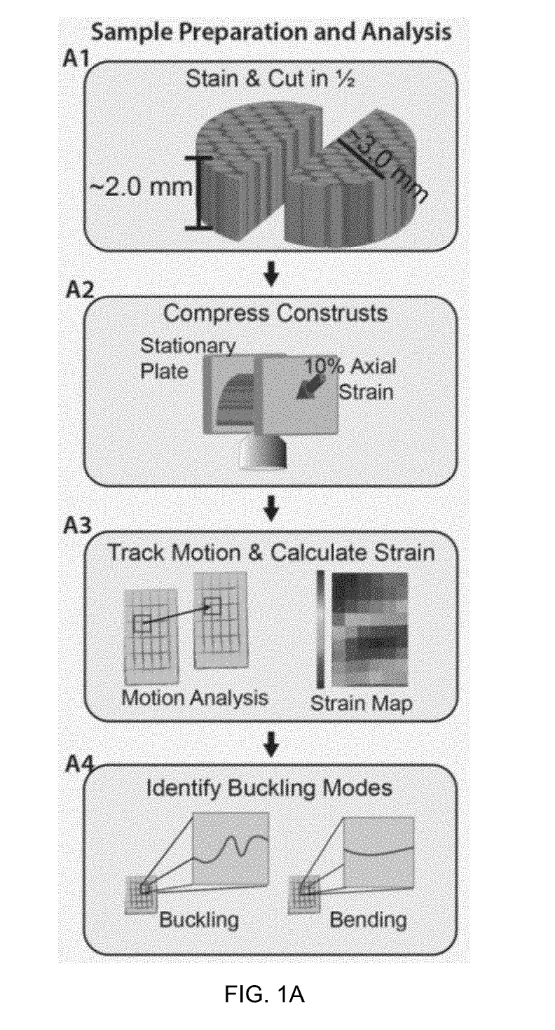

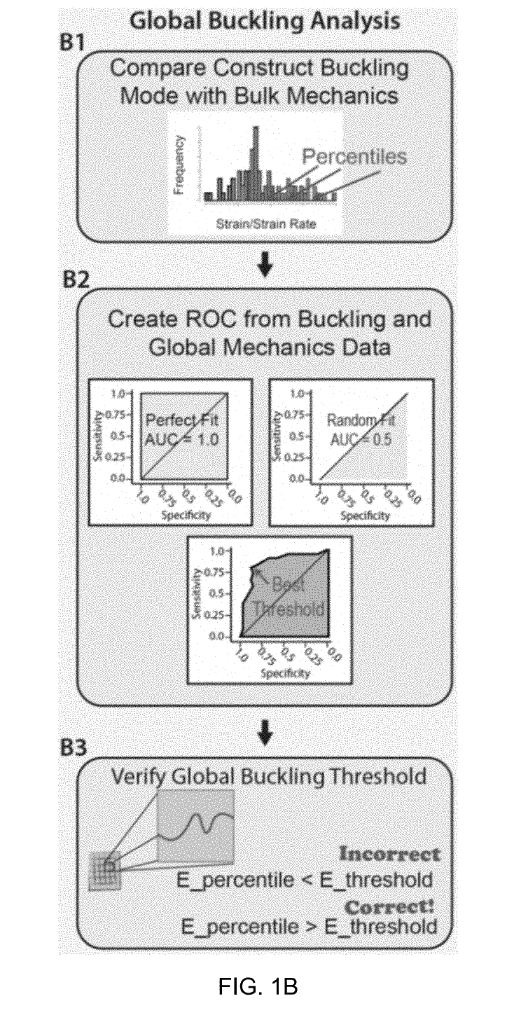

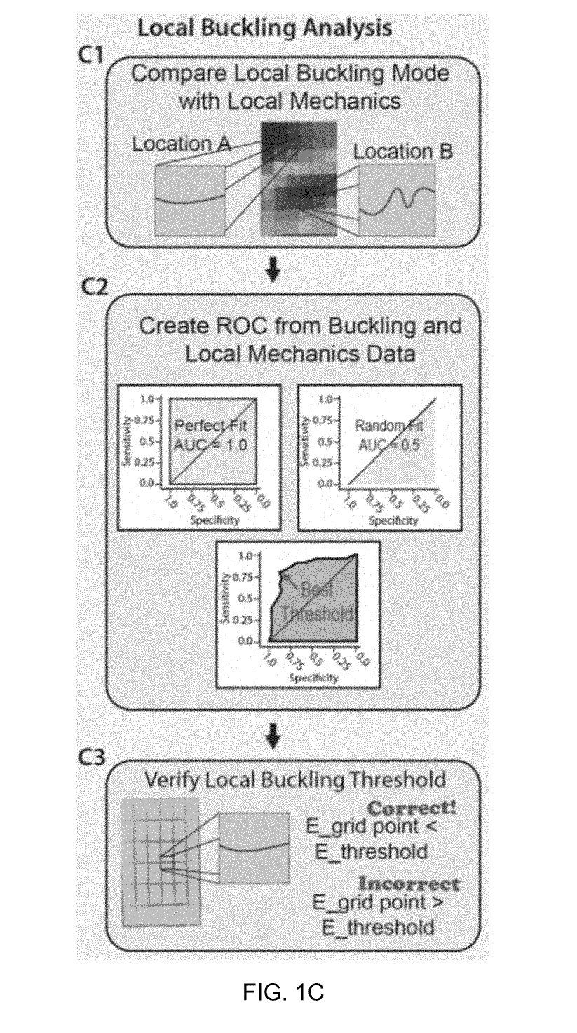

The compressive modulus of constructs was obtained using a modified version of a previously established protocol (Buckley et al., “High-Resolution Spatial Mapping of Shear Properties in Cartilage,” J. Biomech. 43:796-800 (2010) and Buckley et al., “Mapping The Depth Dependence of Shear Properties in Articular Cartilage,” J. Biomech. 41:2430-37 (2008), which are hereby incorporated by reference in their entirety). Briefly, constructs were bisected longitudinally into hemi-cylinders then stained with 14 μg/ml 5-dichlorotriazinyl-aminofluorescein (5-DTAF, Molecular Probes1, Grand Island, N.Y.) for 30 minutes followed by a 20 minute rinse in PBS with protease inhibitors (FIG. 1A (A1)). Constructs were mounted between two plates on a tissue deformation imaging stage (TDIS, FIG. 1A (A2)) and placed on an inverted Zeiss LSM 510 5 live confocal microscope and imaged using a 488 nm laser. Constructs were compressed to 10% axial strain (FIG. 1A (A2)).

The microscale Lagrange strain measured under compressive loading was determined using digital image correlation (DIC) implemented in MATLAB (FIG. 1A (A3)) (Eberl et al., “Digital Image Correlation And Tracking,” Matlab Central, https://www.mathworks.com/matlabcentral/fileexchange/12413-digital-image-correlation-and-tracking (2010) and Jones et al., “Documentation for Matlab-based DIC code 1-42 (2015); Blaber, et al., “Ncorr: Open-Source 2D Digital Image Correlation,” Matlab Software. Exp. Mech. 55, 1105-1122 (2015), which are hereby incorporated by reference in their entirety). The software was set to track local deformation fields on a 78.7 μm grid with a correlation area of 160×160 μm. The microscale axial, transverse, and shear strains at each point were calculated by interpolating the microscale displacements using a finite element shape function. Depth-dependent strain data was calculated by averaging all strain values at a given depth.

### Example 3

### Buckling Identification

Since seeing local buckling in these constructs was expected, bending and buckling were distinguished visually (FIG. 1A (A4)). All observed deformation was elastic. However, bending is characterized by a single long-wavelength arc spanning the length of the image. Buckling is visually classified as a complete sine wave with a small wavelength, less than 400 μm (initial pore size ranged from 200 to 400 μm). The visual characterization of these constructs was then used to establish quantitative differences between buckling and bending. To understand general trends and detailed responses, construct buckling and mechanical behavior was compared on the global and local scale respectively.

### Example 4

### Global Buckling Identification

Global buckling analysis was used to establish a connection between the distribution of construct strains and buckling. A construct was considered buckled based on a threshold identified by examining the strain and strain rate histograms. To accomplish this, a training data set of 8 constructs (4 with buckling and 4 without buckling) was used in a statistical model to identify a threshold to apply to remaining constructs. This model used receiver operating characteristic (ROC) curves to identify a threshold that can distinguished between buckling and bending in constructs. First, histograms of the 3 strains (axial, transverse, and shear) and 3 strain rates (axial, transverse, and shear) were created from the training data set (FIG. 1B (B1)). Because buckling is more likely to occur at higher strains, the strain and strain rate values at 3 percentiles (50^{th}, 75^{th}, and 85^{th}) were recorded. These values were then placed into a receiver operating characteristic (ROC) curve (Streiner et al., “What's Under the ROC? An Introduction to Reciever Operating Characteristics Curves,” Can. J. Psychiatry 52:121-28 (2007), which is hereby incorporated by reference in its entirety) such that a total of 18 curves were created (3 strain and 3 strain rate at each of the 3 percentiles, FIG. 1B (B2)). In a ROC curve the sensitivity (y axis) indicates the ability of the threshold value to correctly predict buckling and the specificity (x axis) indicates the ability of the threshold value to correctly predict bending. The area under the curve (AUC) for each ROC curve was used to determine the percentile and strain or strain rate values that created the best ROC curve. An AUC equal to one indicates the data fits the ROC curve perfectly and an AUC equal to 0.5 indicates a random data fit. Once the best ROC curve fit was determined, the best threshold value was established using the ‘closest to the top left’ method (Streiner et al., “What's Under the ROC? An Introduction to Reciever Operating Characteristics Curves,” Can. J. Psychiatry 52:121-28 (2007), which is hereby incorporated by reference in its entirety). The resulting threshold was applied to all remaining constructs to determine if a construct underwent buckling or bending. The validity of this technique was checked by visually inspecting for buckling or bending. The difference between using the buckling threshold and visually inspecting the constructs was recorded (FIG. 1B (B3)).

### Example 5

### Local Buckling Identification

To complement the global buckling analysis, the local relationship between strain and buckling was analyzed to provide a more detailed understanding of the relationship between strain and buckling. The local buckling analysis identified a local buckling threshold using ROC curves, then applied this threshold to remaining constructs. First, 6 constructs (3 constructs that visually exhibited buckling and 3 constructs that visually exhibited bending) were chosen for analysis. Three strain (axial, transverse, and shear) and 3 strain rates (axial, transverse, and shear) were recorded at each DIC grid point on each of the 6 constructs (FIG. 1C (C1)). The strain and strain rate values were recorded at 4 global compressive strains (2.5%, 5%, 7.5%, and 10%). Each grid point on the 6 constructs was visually labeled as buckling (Y) or bending (N) using visual analysis. The bending or buckling mode and the strain/strain rate at each grid point was used to create six ROC curves (FIG. 1C (C2)). Once the best ROC curve was determined, the best threshold value was identified using the ‘closest to the top left’ method (FIG. 1C (C2)). The threshold value then was applied to all constructs at 4 global compressive strains. This technique's validity was tested by comparing the results of a visual inspection on an additional 2 constructs to the results from the local threshold (FIG. 1C (C3)).

### Example 6

### GAG Analysis

After confocal imaging, constructs were analyzed for GAG content as a measure of cartilage matrix synthesis. Constructs were weighed, lyophilized, and weighed again to obtain construct weight. Then, constructs were papain digested at 60° C. for 14 hours. Sulfated GAG content was measured using a dimethylmethylene blue (DMMB) assay (Enobakhare et al., “Quantification of Sulfated Glycosaminoglycans in Chondrocyte/Alginate Cultures, by Use of 1,9-Dimethylmethylene Blue,” Anal. Biochem. 243:189-91 (1996), which is hereby incorporated by reference in its entirety).

### Example 7

### Statistical Analysis

A Kaplan-Meier survival analysis was used to determine the probability that any grid point in a construct underwent buckling based on application of the local buckling threshold. All correlations were determined using a linear regression. Regressions were considered significant with p<0.05.

### Example 8

### Compression of Constructs

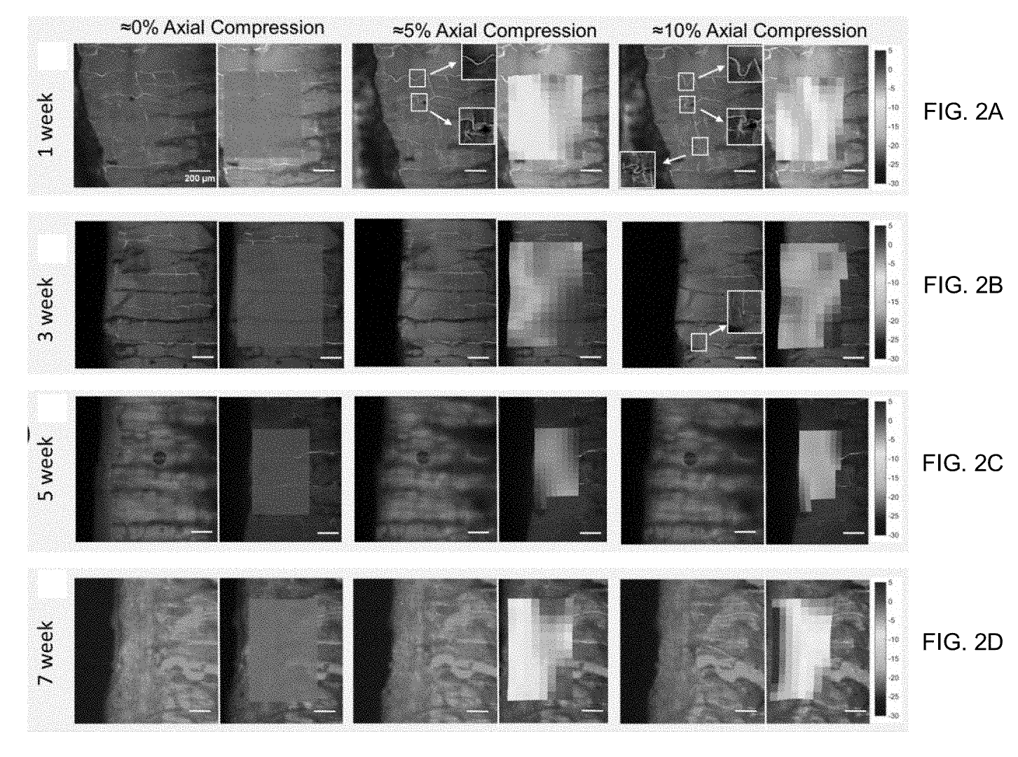

How the construct microscale compressive properties progressed with increased culture was first investigated. Videos were recorded of constructs during compression to 10% axial strain. Video analysis revealed buckling in 1 week constructs at both 5% and 10% global axial compression (FIG. 2A). The buckled areas occurred at random depths from the construct surface. High axial strain occurred at these random depths (Eₓₓ≈20-25%) as seen in the 1 week construct strain maps.

As the constructs matured, buckling decreased and the global axial strain at which this buckling mode occurred increased. In 3 week constructs, buckling occurred at 10% global axial strain (FIG. 2B). The buckled areas still occurred at random depths with areas of high axial strain (Eₓₓ≈20-25%). Constructs grown for 5 weeks buckled less, indicating a transition to bending (FIG. 2C). After 7 weeks, constructs did not buckle, even at 10% axial strain (FIG. 2D). The surface of these constructs exhibited higher axial strain than the deeper portions in the tissue (7 week construct strain maps).

### Example 9

### Global Analysis of Buckling

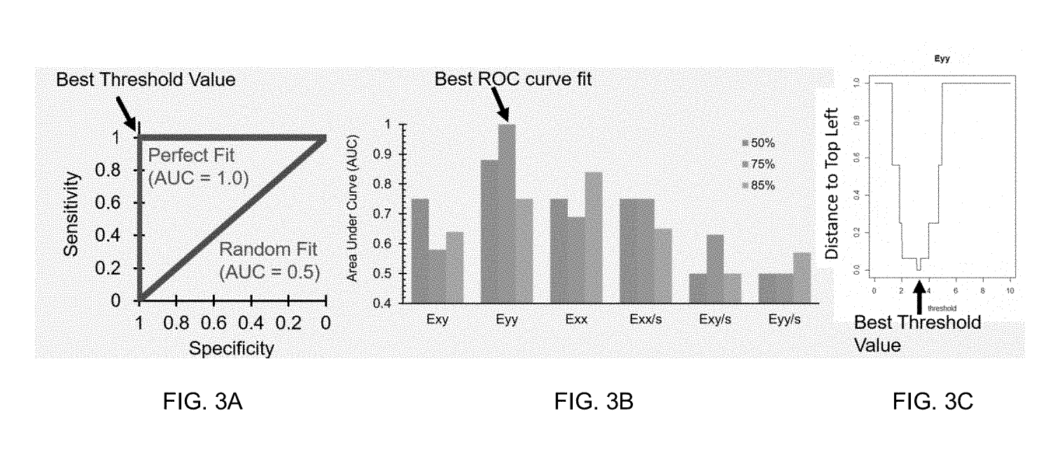

A buckling threshold was established to quantitatively identify buckled constructs. An ROC curve using histogram strain and strain rate percentiles was used to identify this global buckling threshold (FIG. 3A). The transverse strain (E_{yy}) at the 75^{th} percentile provided the highest AUC (AUC=1, FIG. 3B) and, therefore, best distinguishes between a construct with bending and a construct with buckling. A threshold value of E_{yy}=3.2% was chosen using the ‘closest to the top left’ method (FIG. 3C). Therefore, if the transverse strain at the 75^{th} percentile was greater than 3.2% the construct was considered buckled. This buckling threshold provided exact correlation in the training data set. This threshold was applied to all remaining constructs and verified by visual inspection. All 1 and 7 week constructs were correctly identified. Overall, 77% of the 22 constructs were correctly identified as buckled (Table 1).

TABLE 1
Global Buckling Analysis

| | 1 Wk | | 3 Wk | | 5 Wk | | 7 Wk | | Total | |
|---|---|---|---|---|---|---|---|---|---|---|
| | T | V | T | V | T | V | T | V | T | V |
| Number | 4 | 4 | 4 | 3 | 2 | 0 | 0 | 0 | 10 | 7 |
| Buckled | | | | | | | | | | |

| | | | | | |
|---|---|---|---|---|---|
| False | 0 | 2 | 2 | 0 | 4 |
| Positives | | | | | |
| False | 0 | 1 | 0 | 0 | 1 |
| Negatives | | | | | |
| Correct | 100% | 50% | 60% | 100% | 77% |
| (%) | | | | | |

T = “Threshold”;
V = “Visual”

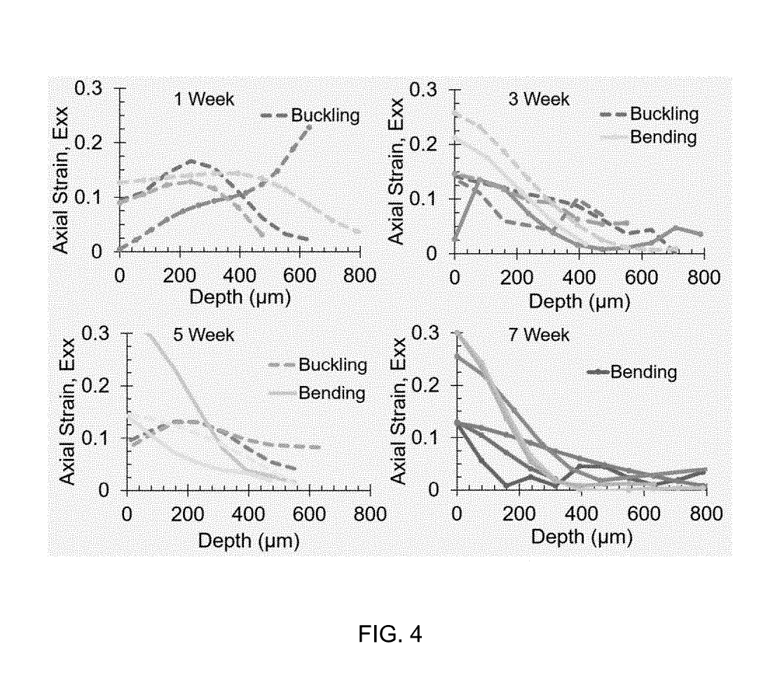

Based on the compressive videos, buckling was believed to correlate with areas of high axial strain. Therefore, the depth dependent axial strain of each construct was plotted and construct was identified as undergoing either buckling or bending on this plot using the global buckling threshold. In 1 week constructs, high axial strain occurred at random tissue depths (FIG. 4A). These areas of high axial strain were associated with buckled locations. All 1 week constructs exhibited buckling. After 3 weeks, the location of high axial strain began to shift toward the surface of the construct (FIG. 4B). Three of the five 3 week constructs exhibited buckling. Similarly, at 5 weeks, areas of high axial strain continued to shift toward the construct surface (FIG. 4C). Two of the five 5-week constructs exhibited buckling. In 7 week constructs, areas of high axial strain were concentrated on the surface (FIG. 4D). Zero 7 week constructs exhibited buckling. Using this analysis, a shift in the areas of high axial strain was observed. In buckled constructs, high strain occurred at random depths while constructs with only bending exhibited high strain at the construct surface.

### Example 10

### Local Analysis of Buckling

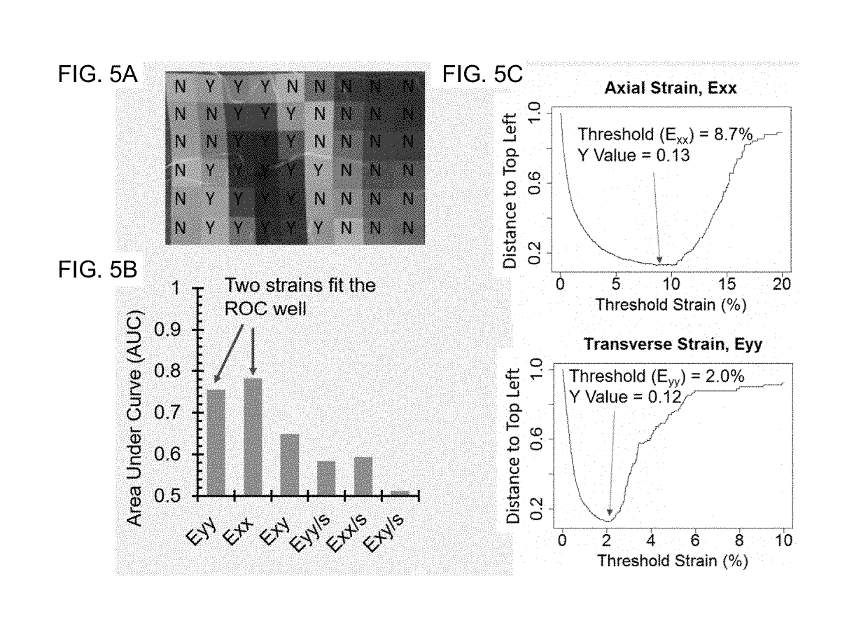

Another quantitative buckling measure was established to investigate the mechanics at buckled locations using ROC curves containing local strain and strain rates. Two strains, axial strain (Eₓₓ) and transverse strain (E_{yy}), fit the ROC well with similar high AUC (AUC_{Eyy}=0.76, AUC_{Exx}=0.78, FIG. 5B). The remaining 4 parameters (E_{xy}, Ėₓₓ, Ė_{xy}, and Ė_{yy}) fit the ROC curves poorly (AUC≈0.5 to 0.7). Therefore, the two best fit ROC curves (Eₓₓ and E_{yy}) were analyzed to identify the best threshold value. The best threshold value for local buckling was the transverse strain, E_{yy}=2.0%, because the shortest distance to the top left on the transverse strain ROC curve (0.12) was less than the shortest distance to the top left (0.13) on the axial strain ROC curve (FIG. 5C). The buckling threshold, E_{yy}=2.0% was applied to all grid points on remaining constructs, such that any grid point with a transverse strain greater than 2.0% was considered buckled. This local buckling threshold was verified on two constructs (one with buckling and one with bending). The threshold correctly identified 85% of all grid points on the two additional constructs (Table 2).

TABLE 2
Local Buckling Analysis

Total

| | Threshold | Visual |
|---|---|---|
| Buckled | 15.1% | 10.7% |

| | |
|---|---|
| False Positives | 48.7% |
| False Negatives | 10.7% |
| Correct | 85.1% |

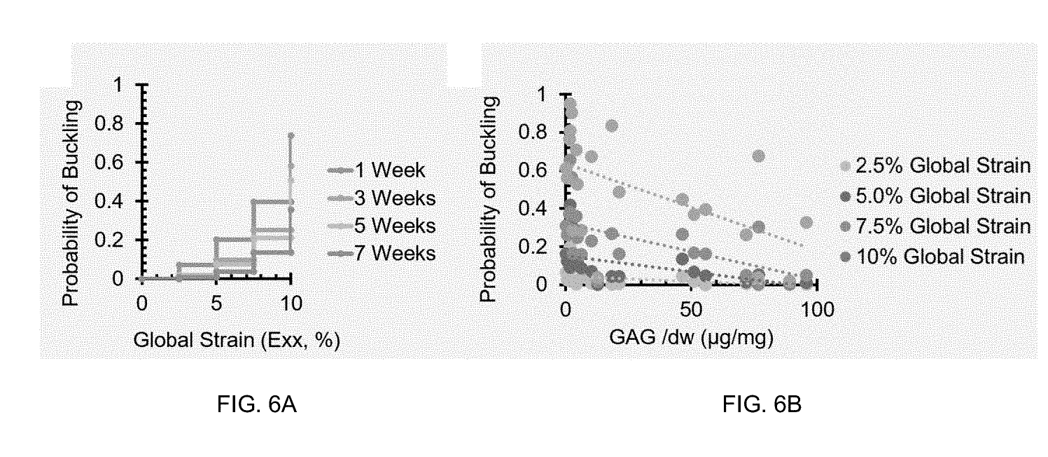

The results from the local buckling analysis can predict the likelihood of buckling at any grid point in a construct. A Kaplan-Meier survival function found the probability that any point in a construct buckled. As expected, this probability was highest after 1 week in culture and lowest after 7 weeks in culture at every global axial strain value (FIG. 6A). One week constructs were two times more likely to exhibit bulking than 7 week constructs.

After axial testing, the probability of buckling was related to a biological construct maturation parameter: GAG content. The GAG content, measured using DMMB, was plotted against the probability that any grid point on the construct buckled (FIG. 6B). At all four global strains the probability of buckling was negatively correlated with construct GAG content. Constructs with low GAG content (<10 μg/mg) had a high probability of buckling especially at 10% axial strain (probabilities ranging from 25% to 95%). Constructs with high GAG content (>40 μg/mg) had a low probability of buckling, as low as 2.1% with most below 50%. Increased global strain increased the probability of buckling, while increased maturation decreased the probability of buckling.

### Example 11

### Analysis of Biochemical Composition

To further understand human tissue engineered construct maturity, the biochemical composition was related to construct buckling and the local mechanical properties.

Cells were isolated from cadaveric human knee articular cartilage, seeded into a honeycomb collagen scaffold, and cultured under low oxygen conditions for 1 to 7 weeks (Crawford et al., “Neocart, An Autologous Cartilage Tissue Implant, Compared with Microfracture for Treatment of Distal Femoral Cartilage Lesions: An FDA Phase-II Prospective, Randomized Clinical Trial After Two Years,” J. Bone Joint Surg. Am. 94:979-89 (2012), which is hereby incorporated by reference in its entirety). Local compositional data was obtained using FTIR transmission (Silverberg et al., “Structure-Function Relations and Rigidity Percolation In the Shear Properties of Articular Cartilage,” Biophys. J. 107:1721-30 (2014), which is hereby incorporated by reference in its entirety). To obtain mechanical data, half the construct was imaged using a custom loading device on a confocal microscope (Buckley et al., “Mapping the Depth Dependence of Shear Properties in Articular Cartilage,” J. Biomech. 41:2430-37 (2008), which is hereby incorporated by reference in its entirety). During imaging, constructs were compressed to 10% axial strain. Then, a 0.5 Hz, 1% oscillatory shear strain was applied. Open-source digital image correlation (DIC) software was used to track deformation and calculate strain (Blaber et al., “Ncorr: Open-Source 2D Digital Image Correlation Matlab Software,” Experimental Mechanics 55(6):1105-22 (2015), which is hereby incorporated by reference in its entirety). A buckling threshold was identified using ROC curves, then applied to the remaining constructs. A Kaplan Meier survival function identified the probability of buckling. A mixed random effects model was used to determine statistical significance of the collagen (Col), aggrecan (Ag), tissue type (TisType), their interaction, and the sample's random effects (including buckling) (Equation 1).

$$Random Effects Model . \\ \begin{matrix}Log ( ShearMod ) = A ( Col ) + B ( Ag ) + C ( TisType ) + D ( Col * Ag ) + E ( Col * TisType ) + F ( Ag * TisType ) + G ( Col * Ag * TisType ) + ( 1 \vert Construct ) . & Equation 1\end{matrix}$$

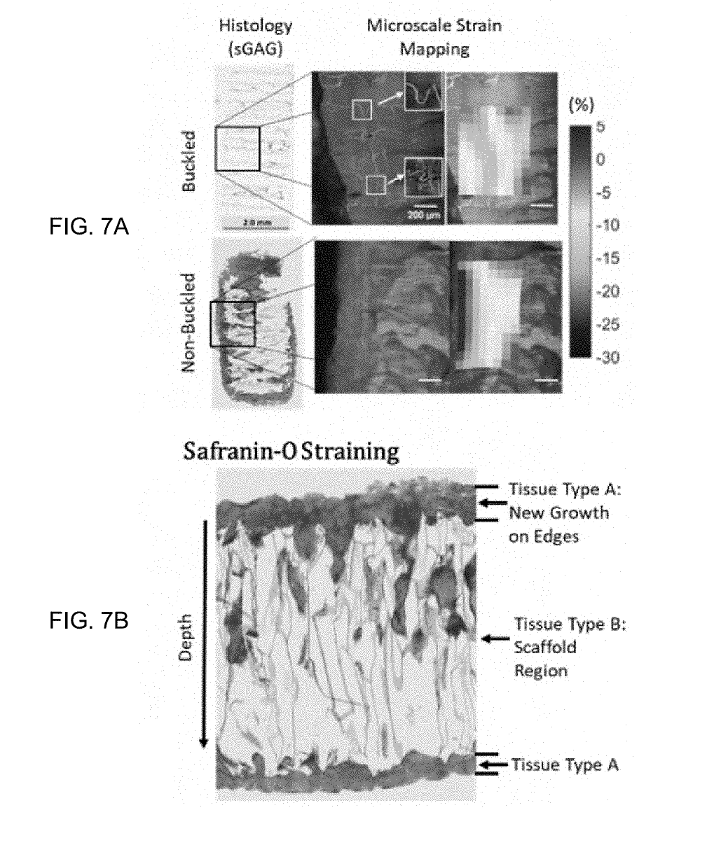

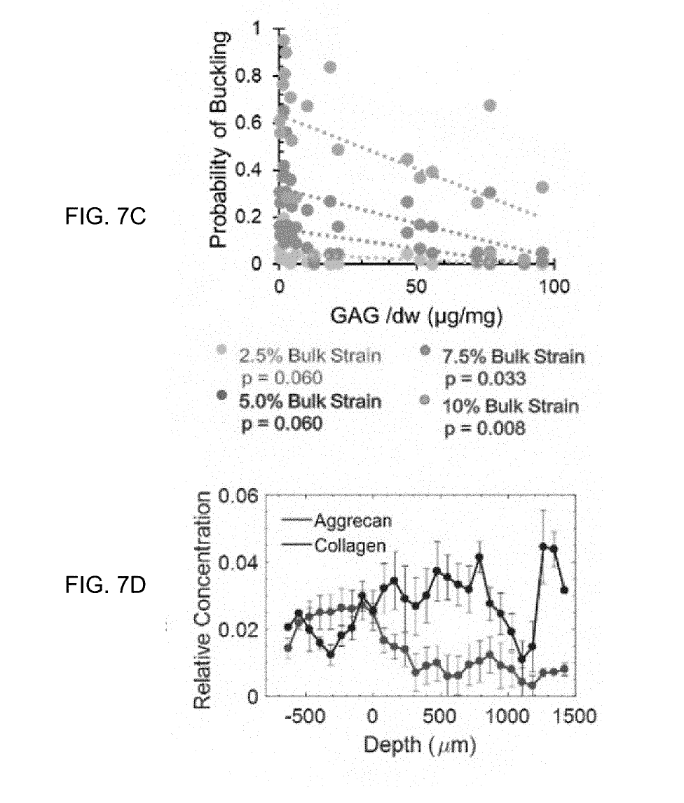

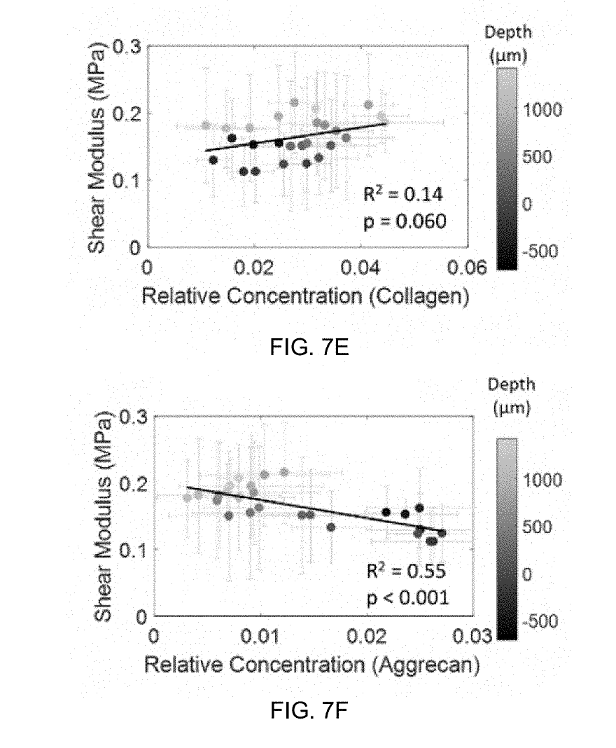

Together, the local mechanics and the biochemical composition provided a new understanding of the function of the scaffold and new matrix. The probability of buckling in a construct decreased with increased total proteoglycan content (FIG. 7C). Collagen content was highest in the middle of the construct (FIG. 7D). Aggrecan content peaked at the interface of the scaffold and the new growth region (FIG. 7D). The shear modulus increased with an increase in collagen concentration, but decreased with increased aggrecan concentration (FIGS. 7E-7F). Further statistical analysis on the relationship between biochemical composition and the local shear modulus revealed the interaction of collagen and aggrecan and the region of biochemical constituents (new growth or scaffold) had the greatest effect on the shear modulus (Table 3). Sample to sample variability decreases in non-buckled samples, when constructs are grouped based on buckling probability (Table 4).

TABLE 3
Model Fit.

| Coefficient | Estimate | P value |
|---|---|---|
| A | −5.42 | 0.2 |
| B | −44.24 | 0.011* |
| C | −2.2 | 4.2E−15 |
| D | 1703 | 0.0013* |
| E | 73 | 1.9E−07* |
| F | 231 | 5.0E−12* |
| G | −8050 | 1.4E−08* |

*p < 0.05

TABLE 4
Sample to Sample Variability.

| | Sample to Sample Variance |
|---|---|
| Buckled | 1.4 |
| Non-Buckled | 0.16 |
| All Samples | 0.89 |

As expected, the global and local shear properties of human tissue engineered cartilage constructs depended on local composition. Specifically, the mechanical contribution of newly synthesized matrix was dependent on where the matrix was deposited. Measurement of the concentration and distribution of deposited matrix enabled the prediction of local mechanics and buckling probability, allowing for a more accurate measurement of construct maturity.

### Example 12

### Discussion

This study identified interactions between local scaffold buckling, local strain, and GAG content in tissue engineered constructs over a range of culture times. During culture, construct behavior under compression changed from buckling at early time points to bending at later time points. Buckling was associated with areas of high strain observed at random depths throughout the constructs. The relationship between the microscale compressive strain, buckling, and the GAG content was also identified in this study. At early time points, constructs with less GAG content were more likely to buckle. In contrast, as the construct matured, the probability of buckling decreased with increased GAG deposition. Collectively, this data suggests that GAG deposition prevents construct buckling and improves the microscale compressive tissue properties.

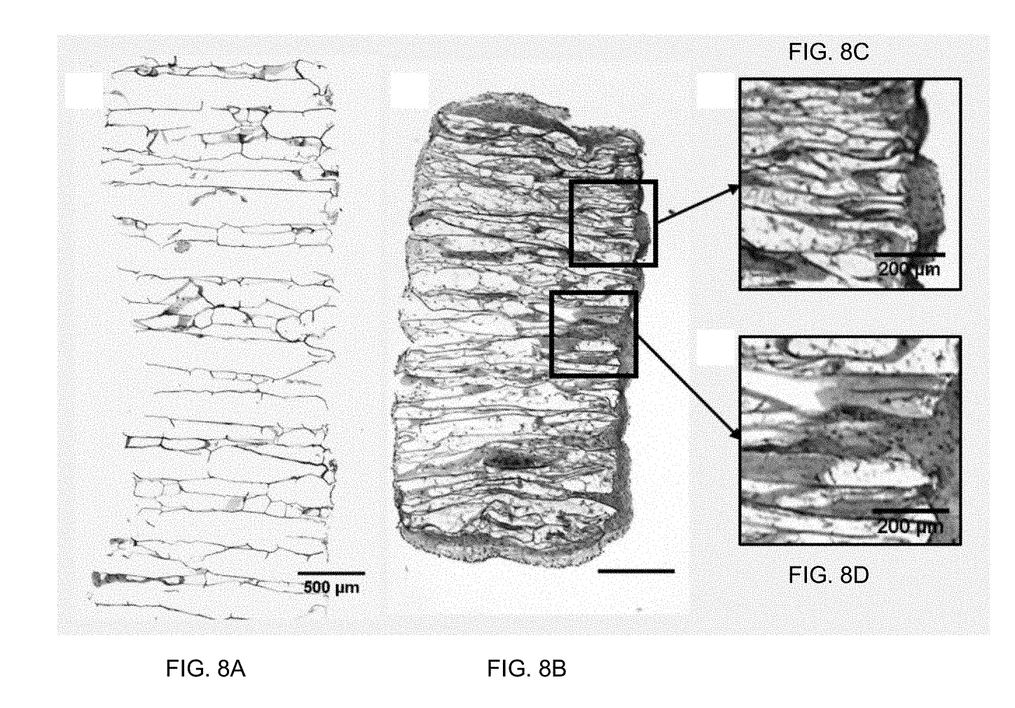

GAG deposition also changed the depth at which maximal axial strain occurred in constructs. Initially, the scaffolds consisted of pores ranging from 200-400 μm (FIG. 8A). This variability in the scaffold structure likely resulted in variations in wall thickness, which in turn created areas of local weakness. During culture, heterogeneous GAG deposition lines the inner scaffold surfaces (FIG. 8C) (Krase et al., “BMP Activation and Wnt-Signalling Affect Biochemistry and Functional Biomechanical Properties of Cartilage Tissue Engineering Constructs,” Osteoarthr. Cartil. 22:284-92 (2014) and Middendorf et al., “Mechanical Properties and Structure-Function Relationships of Human Chondrocyte-Seeded Cartilage Constructs after In Vitro Culture.” J. Orthop. Res. 1-9 (2017), which are hereby incorporated by reference in their entirety), which reinforces the walls enough to overcome local weaknesses. GAG content also concentrates on the outside edges of the construct (FIG. 8B). The highly concentrated, GAG-rich regions on the construct surface are very compliant under compression. The shift in the depth at which high axial strain occurred indicates how variability in the scaffold and localization of GAG deposition could change the construct's mechanical behavior.

To fully understand why the probability of buckling decreased with increased GAG content, classic mechanics buckling theories were considered. Two different theories adequately explain this system. The first theory requires the GAG content and GAG deposition to increase the ratio of pore wall thickness to wall width by lining the inner scaffold surfaces (FIG. 8C), where wall width refers to the distance between the nodes connecting multiple walls. Increasing this ratio increases the strain required for the onset of buckling (Gibson et al., Cellular Materials in Nature and Medicine, Cambridge University Press, Cambridge, UK (2010), which is hereby incorporated by reference in its entirety). Since the onset of buckling is proportional to the square of the ratio of wall thickness to wall width, eventually the construct will never leave the elastic region. This theory requires the GAG and ECM deposition on the inner scaffold walls to have approximately the same modulus as the initial scaffold. The second theory requires the construct to behave similar to a set of tubes (honeycomb scaffold) filled with a compliant core (GAG rich substance, FIG. 8D). The compliant core supports the outer tube under compression, increasing the resistance to buckling (Gibson et al., Cellular Materials in Nature and Medicine, Cambridge University Press, Cambridge, UK (2010), which is hereby incorporated by reference in its entirety). This theory requires a sufficiently stiff inner core to act as an elastic foundation. Both theories provide evidence that might explain why an increase in the total GAG content correlated with a decreased probability of buckling. However a quantitative study on the local GAG and collagen content is necessary to fully understand buckling.

A major challenge to tissue engineered cartilage is understanding the level of construct maturity required for implantation. In both lab experiments and clinical trials maturity is typically assessed using GAG content or global compressive properties. These parameters do not define any distinct changes in microstructural features. The identification of buckling, a distinct on-off phenomenon, can define distinct changes in microstructure and characterize implant maturity. In this study, constructs required an adequate pore fill to prevent buckling at 10% axial strain. Similarly, the location of GAG (FIGS. 8A-8D) with relation to the scaffold is believed to have changed the local mechanics and buckling.

In this study, GAG content and location played an important role in determining the microscale compressive properties of tissue engineered constructs. However, some studies have identified collagen content as an important aspect of tissue engineered cartilage. A large change in collagen content is necessary to improve the compressive properties of tissue engineered cartilage by a small amount (Griffin et al., “Mechanical Properties and Structure-Function Relationships in Articular Cartilage Repaired Using Igf-I Gene-Enhanced Chondrocytes,” J. Orthop. Res. 34:149-53 (2016), which is hereby incorporated by reference in its entirety). A large change in collagen content is unlikely to occur during the short 7 weeks of culture. Therefore, collagen was not measured. Additionally, various growth parameters such as seeding density, scaffold structure, and growth media could change the amount and type of ECM deposited, and the onset of buckling. Seeding density and the growth parameters in this study were chosen to match NeoCart®, a tissue engineered construct in advanced clinical trials. Finally, the relationship between local ECM components and microscale mechanics of constructs is not well understood. FTIR or Raman spectroscopy can determine the relationship between local ECM (Kunstar et al., “Label-Free Raman Monitoring of Extracellular Matrix Formation in Three-Dimensional Polymeric Scaffolds,” J. R. Soc. Interface 10:20130464 (2013) and Rieppo et al., “Fourier Transform Infrared Spectroscopic Imaging and Multivariate Regression for Prediction of Proteoglycan Content of Articular Cartilage,” PLoS One 7(2):e32344 (2012), which are hereby incorporated by reference in their entirety) and local mechanics (Silverberg et al., “Structure-Function Relations and Rigidity Percolation in the Shear Properties Of Articular Cartilage,” Biophys. J. 107:1721-30 (2014), which is hereby incorporated by reference in its entirety).

Changes in the microscale compressive properties of human chondrocyte-seeded collagen constructs during maturation were measured. Results indicate the amount and location of GAG content influence buckling. Since the constructs tested in this study are similar to a tissue engineered construct (NeoCart®) that is currently in advanced human trials (Crawford et al., “Neocart, An Autologous Cartilage Tissue Implant, Compared with Microfracture for Treatment of Distal Femoral Cartilage Lesions: An FDA Phase-II Prospective, Randomized Clinical Trial After Two Years,” J. Bone Joint Surg. Am. 94:979-89 (2012) and Crawford et al, “An Autologous Cartilage Tissue Implant Neocart for Treatment of Grade III Chondral Injury to the Distal Femur: Prospective Clinical Safety Trial at 2 Years,” Am. J. Sports Med. 37:1334-43 (2009), which are hereby incorporated by reference in their entirety), the study provided insight regarding the microscale mechanical properties that change the global function of a successful implant. Allowing adequate ECM deposition prior to implantation prevents buckling. Prevention of buckling improves the compressive properties and global function of human constructs.

Local biochemical composition in addition to the global biochemical composition can be used to predict buckling. Methods of measuring local biochemical composition include Fourier Transform Infrared Spectroscopy, Raman Spectroscopy, and Near Infrared Spectroscopy (NIR), etc. Buckling is a nonlinear phenomenon that causes high amounts of sample to sample variability (Table 4). The local biochemical information and statistical analysis shown here shows evidence that new matrix (collagen and proteoglycans) deposited in the scaffold increases the local modulus more than new matrix on the scaffold periphery (Table 3). This increase in local modulus helps to reduce local construct buckling.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of determining if a tissue engineered construct is ready for implantation, said method comprising:
providing a tissue engineered construct comprising a scaffold having pores;
analyzing the tissue engineered construct for buckling of pores; and
determining whether the tissue engineered construct is ready for implantation based on said analyzing.

2. The method according to claim 1, wherein the tissue engineered construct is selected from the group consisting of a tissue engineered cartilage construct, a tissue engineered bone construct, a tissue engineered tendon construct, a tissue engineered ligament construct, and a tissue engineered heart valve construct.

3. The method according to claim 1, wherein the tissue engineered construct is a human tissue engineered construct.

4. The method according to claim 1, wherein said providing comprises:
seeding cells onto a scaffold; and
culturing the cells to form a tissue engineered construct.

5. The method according to claim 1, wherein said analyzing comprises:
compressing the tissue engineered construct;
detecting whether pores of the scaffold of the tissue engineered construct buckled.

6. The method according to claim 5, wherein said compressing comprises:
staining the tissue engineered construct;
mounting the tissue engineered construct on a tissue deformation imaging stage; and
compressing the constructs using axial compression.

7. The method according to claim 5, wherein said detecting comprises:
measuring microscale axial, transverse, and shear strains and axial, transverse, and shear strain rates on a grid using digital image correlation;
identifying a buckling threshold; and
applying the buckling threshold to classify the tissue engineered construct as buckled or not buckled.

8. The method according to claim 5, wherein said determining comprises:
characterizing the tissue engineered construct as ready for implantation if no buckling of the pores of the scaffold of the tissue engineered construct was detected.

9. The method according to claim 1, wherein said analyzing comprises:
measuring bulk proteoglycan content; and
determining the probability that the tissue engineered construct will buckle when compressed based on the bulk proteoglycan content.

10. The method according to claim 9, wherein said measuring comprises:
obtaining the construct weight;
digesting the construct; and
determining sulfated proteoglycan content as a proportion of the construct weight using a dimethylmethylene blue assay.

11. The method according to claim 10, wherein said determining the probability that the tissue engineered construct will buckle when compressed based on the bulk proteoglycan content comprises:
characterizing the tissue engineered construct as having a probability of buckling of 20% or less if the bulk proteoglycan content is 75 μg/mg (dry weight) or more.

12. The method according to claim 9, wherein said determining comprises:
characterizing the tissue engineered construct as ready for implantation if the probability that the tissue engineered construct will buckle when compressed is 25% or less.

13. The method according to claim 1, wherein said determining comprises:
determining whether the tissue engineered construct is ready for human implantation.

14. The method of claim 1, wherein said providing comprises providing a plurality of tissue engineered constructs and said analyzing involves analyzing one or more, but not all, of the plurality of tissue engineered constructs.

15. A method of determining if a tissue engineered construct is ready for implantation, said method comprising:
providing a tissue engineered construct comprising a scaffold having pores;
analyzing the tissue engineered construct in a non-destructive manner; and
determining whether the tissue engineered construct is ready for implantation.

16. The method according to claim 15, wherein the tissue engineered construct is selected from the group consisting of a tissue engineered cartilage construct, a tissue engineered bone construct, a tissue engineered tendon construct, a tissue engineered ligament construct, and a tissue engineered heart valve construct.

17. The method according to claim 15, wherein the tissue engineered construct is a human tissue engineered construct.

18. The method according to claim 15, wherein said providing comprises:
seeding cells onto a scaffold; and
culturing the cells to form a tissue engineered construct.

19. The method according to claim 15, wherein said analyzing comprises:
detecting biochemical compositional data; and
determining the probability of whether pores of the scaffold of the tissue engineered construct will buckle when compressed.

20. The method according to claim 19, wherein the biochemical compositional data is selected from the group comprising: total proteoglycan content, total collagen content, total aggrecan content, microscale proteoglycan content, microscale collagen content, microscale aggrecan content, and combinations thereof.

21. The method according to claim 19, wherein said measuring is carried out using vibrational spectroscopy.

22. The method according to claim 19, wherein said determining comprises:
characterizing the tissue engineered construct as having a probability of buckling of 20% or less if the bulk proteoglycan content is 75 μg/mg (dry weight) or more.

23. The method according to claim 15, wherein said determining comprises:
characterizing the tissue engineered construct as ready for implantation if the probability that the pores of the scaffold of the tissue engineered construct will buckle when compressed is less than 25%.

24. The method according to claim 15, wherein said determining comprises:
determining whether the tissue engineered construct is ready for human implantation.
